(19) United States
(12) Patent Application Publication
(10) Pub. No.: US 2012/0095264 A1
(43) Pub. Date: Apr. 19, 2012

(54) SOLID STATES OF ALISKIREN FREE BASE

(75) Inventors: Nina Finkelstein, Herzliya (IL); Boris Finkelstein, Herzliya (IL); Ariel Mittelman, Elad (IL)

(73) Assignee: TEVA Pharmaceutical Industries Ltd., Petah Tiqva (IL)

(21) Appl. No.: 12/994,072

(22) PCT Filed: Jun. 5, 2009
(86) PCT No.: PCT/US2009/046398

§ 371 Date: Aug. 11, 2011

Related U.S. Application Data
(60) Provisional application No. 61/059,617, filed on Jun. 6, 2008.
(60) Provisional application No. 61/092,172, filed on Aug. 27, 2008.

(51) Int. Cl. C07C 237/20

(52) U.S. Cl. 564/157

(57) ABSTRACT

The present invention describes a solid state of aliskiren free base, and process for the preparation thereof.

### CROSS-REFERENCE TO PRIORITY APPLICATION

This application is a national stage of International Application PCT/US2009/046398, filed Jun. 5, 2009, which claims benefit of U.S. Provisional Patent Application Nos. 61/059,617, filed Jun. 6, 2008, and 61/092,172, filed Aug. 27, 2008, the contents of which are incorporated herein in their entirety by reference.

### FIELD OF THE INVENTION

The present invention relates to a solid state of aliskiren free base, its amorphous form, and process for the preparation thereof.

### BACKGROUND OF THE INVENTION

Aliskiren hemifumarate (CAS Registry Number: 173334-58-2), having the chemical name: (2S,4S,5S,7S)-N-(2-carbamoyl-2-methylpropyl)-5-amino-4-hydroxy-2,7-diisopropyl-8-[4-methoxy-3-(3-methoxypropoxy)phenyl]octanamide hemifumarate (C₃₀H₅₃N₃O₆.0.5 C₄H₄O₄) is indicated for treatment of hypertension, acting as a renin inhibitor, and marketed by Novartis as TEKTURNA® as a once-daily formulation. The free base form of aliskiren, (2S,4S,5S,7S)-N-(2-carbamoyl-2-methylpropyl)-5-amino-4-hydroxy-2,7-diisopropyl-8-[4-methoxy-3-(3-methoxypropoxy)phenyl]octanamide, can be described according to the following formula:

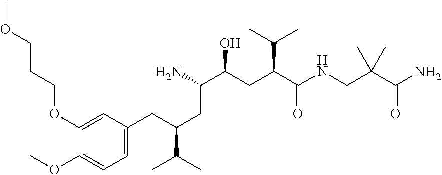

Synthesis of aliskiren and its related compounds are referred to in U.S. Pat. No. 5,559,111, while pharmacological actions, pharmacokinetics and clinical studies of aliskiren and its related compounds are referred to in Lindsay, K. B. et. al., J. Org. Chem., Vol. 71, pp 4766-4777 (2006) and in Drugs of the Future, Vol. 26, No. 12, pp 1139-1148 (2001).

U.S. Pat. No. 5,559,111 refers to the preparation of a crystalline faun of aliskiren hemifumarate having a melting point of about 95-104° C. by crystallizing from an ethanol/acetonitrile mixture in a 1 to 19 volume ratio and then drying at 60° C. U.S. Pat. No. 6,730,798 refers to the preparation of aliskiren hemifumarate using hydrogenation of an aliskiren derivative. Preparation of aliskiren hemifumarate from aliskiren hydrochloride is also described in U.S. Pat. No. 5,559,111 and US2006/0154926.

The discovery of new solid states of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. Thus, there is a need in the art for new forms of pharmaceutically useful compounds of aliskiren.

### SUMMARY OF THE INVENTION

The present invention encompasses solid aliskiren free base. The present invention further encompasses an amorphous form of aliskiren free base.

The present invention further encompasses a process for preparing the solid (including amorphous) aliskiren free base comprising providing a solution of aliskiren free base in a solvent selected from esters having low boiling point, diethyl ether, diisopropyl ether, isopropanol (IPA) and dichloromethane; and removing the solvent to obtain the solid (including amorphous) aliskiren free base. Preferably, the obtained aliskiren free base is in an amorphous form.

The solid (including amorphous) aliskiren free base of the present invention can be used for the manufacture of a medicament, preferably for the treatment of hypertension.

The present invention includes the use of a solid aliskiren free base for the manufacture of an aliskiren salt, preferably aliskiren hemifumarate. Thus, the present invention encompasses a process for preparing aliskiren salt, preferably an aliskiren hemifumarate salt, comprising obtaining solid (including amorphous) aliskiren free base according to the process of the present invention and further converting the obtained aliskiren free base to an aliskiren salt.

### BRIEF DESCRIPTION OF THE FIGURES

FIG. 1 represents a powder XRD pattern of amorphous aliskiren free base. The three peaks that appear in the diffractogram are the result of contamination that is not relevant to aliskiren.

FIG. 2 represents a powder XRD pattern of amorphous aliskiren free base obtained according to example 3.

### DETAILED DESCRIPTION OF THE INVENTION

Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in an organic solvent. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state faun of a compound may also affect its behavior on compaction and its storage stability.

These practical physical characteristics are influenced by the conformation and orientation of molecules in the unit cell, which define a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

One of the most important physical properties of a pharmaceutical compound, which can form polymorphs or solvates, is its solubility in organic solvents, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

As used herein, “isolated” refers to a compound being physically separated from the reaction mixture. For example, the separation can be done by elution from an HPLC column and further drying the compound.

As used herein, “reduced pressure” refers to a pressure of below atmospheric pressure, i.e., a pressure of less than 1 atm. Reduced pressure may be obtained for example, by vacuum. Vacuum refers to a pressure of less than 100 mm Hg.

The present invention addresses a need in the art for obtaining solid aliskiren base. While removal of the solvent (for example, by evaporation) from a solution of aliskiren base typically results in a non-isolated residue, preferred processes of the present invention result in an isolated solid aliskiren free base.

As used herein, “room temperature” refers to a temperature of about 15° C. to about 30° C., preferably less about 15° C. to about 25° C. and more preferably about 20° C. to about 25° C.

As used herein “low boiling point esters” refer to esters having a boiling point between about 30° C. to about 90° C. Examples of low boiling point esters that may be used in the present application include methyl acetate, ethyl acetate, methyl formate, propyl formate and ethyl formate.

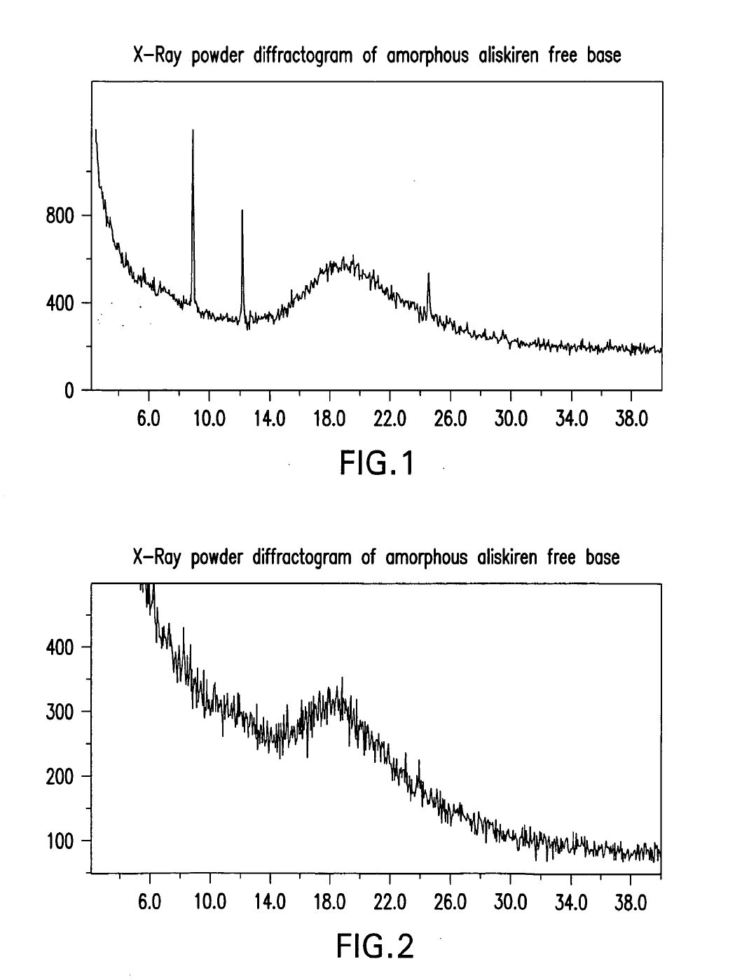

Aliskiren free base may be analyzed to determine the nature of the product. The X-ray powder diffraction pattern of amorphous aliskiren free base does not exhibit peaks characteristic of crystal forms of aliskiren free base, demonstrating the amorphous nature of the product. The presence of characteristic peaks for crystalline forms would indicate the presence of a crystalline form of aliskiren free base. The three peaks that appear in the diffractogram of FIG. 1 are the result of contamination that is not relevant to crystalline forms of aliskiren.

In one embodiment, the invention encompasses aliskiren free base in a solid form.

In another embodiment, the invention encompasses aliskiren free base in an amorphous form, as characterized by the X-ray powder diffraction pattern depicted in FIGS. 1 and 2.

The solid aliskiren free base may be prepared by a process comprising providing a solution of aliskiren free base in a solvent selected from esters having low boiling point, diethyl ether, diisopropyl ether, isopropanol (IPA) and dichloromethane; and removing the solvent to obtain the solid aliskiren free base.

The obtained aliskiren free base is preferably in an amorphous form.

Preferably, the aliskiren free base starting material is used as oil.

Typically, removal of the solvent is performed by evaporation, preferably, under reduced pressure, or vacuum.

The low boiling point esters used in the process described above can be methyl acetate, ethyl acetate, methyl formate, propyl formate and ethyl formate.

Preferably, the solvents used in the process are selected from a group consisting of methyl acetate, ethyl acetate, IPA and dichloromethane, more preferably it is ethyl acetate or dichloromethane.

When IPA is used as a solvent, an additional gradual cooling step is preferably performed. The evaporated residue is preferably first cooled to a temperature of about 10° C. to about −10° C. for about 3 to 5 days, then to a temperature of about −10° C. to about −40° C. for about 2 to 4 days, further to a temperature of about −40° C. to about −80° C. for about 0.5 to 2 days, and finally letting the temperature reach a temperature of about −10° C. to about −30° C. for about 15 to 20 days. Most preferably, the reaction mixture is preferably first cooled to a temperature of about 0° C. for about 4 days, then to a temperature of about −20° C. for about 3 days, further to a temperature of about −78° C. for about 1 day, and finally letting the temperature get to about −20° C. for about 14 days.

Typically, the solution is obtained at room temperature.

The aliskiren base and the solvent are preferably used in a ratio of about 1:2 to about 1:20 (w/v) of grams aliskiren base to mls of solvent, more preferably in about 1:5 to about 1:15 (w/v) and most preferably, in about 1:5 to about 1:10 (w/v).

The evaporation is preferably performed for about 5 minutes to about 30 minutes, more preferably for about 5 minutes to about.

The evaporation is preferably performed at a temperature of not more than about 40° C.

The aliskiren free base starting material can be prepared by any method known in the art. For example, aliskiren free base is obtained as a non-isolated evaporation residue in the conversion process from aliskiren hydrochloride to aliskiren hemifumarate described in U.S. Pat. No. 5,559,111, or according to reference example 1 of the present application wherein aliskiren free base is prepared by a process comprising providing a solution of aliskiren hemifumarate in water, adding a base (e.g., aqueous ammonia); extracting aliskiren free base with an organic solvent (e.g., ethyl acetate) at a temperature of about 40° C. to 70° C. to obtain a two-phase system; and recovering the aliskiren free base from the organic phase.

The present invention further encompasses 1) a pharmaceutical composition comprising the solid aliskiren free base described above and at least one pharmaceutically acceptable excipient, and 2) the use of the above-described solid aliskiren free base, for the manufacture of a pharmaceutical composition, wherein the pharmaceutical composition can be useful for the treatment of hypertension.

The pharmaceutical composition of the present invention can be in a solid or a non-solid form. If the pharmaceutical composition is in a non-solid faun, the solid aliskiren free base in the composition can present as a solid in the non-solid pharmaceutical composition, e.g., as a suspension, foam or ointment, etc.

The pharmaceutical composition can be prepared by a process comprising combining the above-described solid aliskiren free base with at least one pharmaceutically acceptable excipient. The solid aliskiren free base can be obtained by any of the processes of the present invention as described above.

The pharmaceutical composition can be used to make appropriate dosage forms such as tablets, powders, capsules, suppositories, sachets, troches and lozenges.

The solid aliskiren free base of the present invention, particularly in a pharmaceutical composition and dosage form, can be used to treat hypertension in a mammal such as a human, comprising administering a treatment effective amount of the solid aliskiren free base in the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

The solid aliskiren free base used in the above-described pharmaceutical composition is preferably in an amorphous form.

The present invention further encompasses a process for preparing aliskiren salt comprising obtaining a solid aliskiren free base according to any of the processes described above and further converting to aliskiren salt. Preferably the obtained aliskiren salt is aliskiren hemifumarate salt.

Conversion of aliskiren free base to aliskiren salt may be obtained according to methods known in the art, for example by combining the solid aliskiren free base with an acid, such as fumaric acid.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to limit its scope in any way.

### EXAMPLES

### Powder XRD (X-Ray Diffraction)

ARL X-ray powder diffractometer model X′TRA-030, Peltier detector, round standard aluminum sample holder with round zero background silicon plate was used. The cathode is CuKa radiation, λ=1.5418 Å. Scanning parameters: Range: 2-40 degrees two-theta continuous Scan, Rate: 3 deg/min.

### Preparation of Aliskiren Free Base

### Reference Example 1

Aliskiren hemifumarate amorphous (0.35 g) was dissolved in 10 ml of water, basified with 25% aqueous ammonia (2 ml) and extracted with ethyl acetate twice (2×15 ml). The combined organic phase was washed with water, dried with anhydrous sodium sulfate. Ethyl acetate was evaporated under vacuum at 40-50° C. to give aliskiren base as an oil (0.26 g).

### Preparation of Amorphous Aliskiren Free Base

### Example 2

A solution of aliskiren base (0.35 g) in 2 ml of isopropanol was stirred at room temperature without precipitation. Isopropanol was evaporated for about 5-15 minutes, under vacuum and aliskiren base was kept at 0° C. for 4 days, then at −20° C. for 3 days, at −78° C. for 1 day and again at −20° C. for 2 weeks to give amorphous aliskiren base.

### Example 3

Aliskiren base (0.5 g) was dissolved in ethyl acetate (5 ml). Ethyl acetate was evaporated for about 5-15 minutes under vacuum at room temperature and dried under vacuum at room temperature overnight to give an off-white powder of amorphous aliskiren base.

### Example 4

Aliskiren base (0.5 g) was dissolved in dichloromethane (5 ml). Dichloromethane was evaporated for about 5-15 minutes under vacuum at room temperature and dried under vacuum at room temperature overnight to give an off-white powder of amorphous aliskiren base.

1. Solid form of aliskiren free base.

2. The solid form of claim 1 wherein the aliskiren free base is in amorphous fatal.

3. The amorphous form of claim 2, characterized by the an X-ray powder diffraction pattern depicted in FIG. 1 and or 2.

4. A process for preparing solid aliskiren free base according to claim 1 comprising providing a solution of aliskiren free base starting material in a solvent selected from the group consisting of esters having low boiling point, diethyl ether, diisopropyl ether, isopropanol and dichloromethane; and removing the solvent to obtain the solid aliskiren free base.

5. The process of claim 4 wherein the obtained solid aliskiren free base is in amorphous form.

6. The process of any of claim 4 or 5 wherein the aliskiren free base starting material is an oil.

7. The process of claim 4 wherein removal of the solvent is performed by evaporation.

8. The process of claim 4 wherein removal of the solvent is performed under reduced pressure.

9. The process of claim 4 wherein removal of the solvent is performed under vacuum.

10. The process of claim 4 wherein the solution is obtained at room temperature.

11. The process of claim 4 wherein the ester is methyl acetate, ethyl acetate, methyl formate, propyl foimate or ethyl formate.

12. The process of claim 4 wherein the solvent is ethyl acetate or dichloromethane.

13. The process of claim 4 wherein the solvent is isopropanol and an additional gradual cooling step is performed on the evaporated residue.

14. The process of claim 13 wherein the cooling is carried out (a) firstly to a temperature of about 10° C. to about −10° C., then (b) to a temperature of about −10° C. to about −40° C., further (c) to a temperature of about −40° C. to about −80° C., and finally (d) to a temperature of about −10° C. to about −30° C.

15. The process of claim 14 wherein the cooling is (a) firstly to a temperature of about 0° C., then (b) to a temperature of about −20° C., further (c) to a temperature of about −78° C., and finally (d) to a temperature of about −20° C.

16. The process of claim 14 wherein step (a) is carried out over about 3-5 days, step (b) is carried out over about 2-4 days, step (c) is carried out over about 0.5-2 days, and step (d) is carried out over about 5-20 days.

17. The process of claim 14 wherein step (a) is carried out over about 4 days, step (b) is carried out over about 3 days, step (c) is carried out over about 1 day, and step (d) is carried out over about 14 days.

18. The process of claim 4 wherein the aliskiren base and the solvent are used in a ratio of about 1:2 to about 1:20 (w/v) of grams aliskiren base to mls solvent.

19. The process of claim 18 wherein the aliskiren base and the solvent are used at a ratio of about 1:5 to about 1:10 (w/v).

20. (canceled)

21. (canceled)

22. A process for preparing aliskiren salt comprising obtaining solid aliskiren free base of any of claims 1 to 3 and converting the obtained aliskiren free base to an aliskiren salt.

23. The process of claim 22 wherein the obtained aliskiren salt is aliskiren hemifumarate.
